(19) United States
(12) Patent Application Publication
(10) Pub. No.: US 2007/0134756 A1
(43) Pub. Date: Jun. 14, 2007

(54) Method and system of verifying protein-protein interaction using text mining

(75) Inventors: Jae Soo Lim, Daejeon-city (KR); Hyun Chul Jang, Daejeon-city (KR); Joon Ho Lim, Daejeon-city (KR); Soo Jun Park, Seoul (KR); Seon Hee Park, Daejeon-city (KR)

Correspondence Address: MAYER, BROWN, ROWE & MAW LLP, 1909 K STREET, N.W., WASHINGTON, DC, 20006, US

(73) Assignee: ELECTRONICS AND TELECOMMUNICATIONS RESEARCH INSTITUTE

(21) Appl. No.: 11/601,620

(22) Filed: Nov. 20, 2006

(30) Foreign Application Priority Data
Dec. 8, 2005 (KR) 10-2005-0119279
Mar. 17, 2006 (KR) 10-2006-0024786

(51) Int. Cl. G06F 19/00; C12Q 1/37

(52) U.S. Cl. 435/23; 702/19

(57) ABSTRACT

Provided are a method and system for verifying a protein-protein interaction according to a text mining method. The method includes extracting protein-protein interaction information from protein-related documents searched for from a bio-information document database, according to a text mining method, mapping the protein-protein interaction information to corresponding ontology identifications, and filtering the mapped protein-protein interaction information according to a frequency of the information and an impact factor of a corresponding protein-related document in order to obtain highly-weighted information.

### CROSS-REFERENCE TO RELATED PATENT APPLICATION

This application claims the priorities of Korean Patent Application No. 10-2005-0119279, filed on Dec. 8, 2005 and Korean Patent Application No. 10-2006-0024786, filed on Mar. 17, 2006, in the Korean Intellectual Property Office, the disclosures of which are incorporated herein in their entirety by reference.

### BACKGROUND OF THE INVENTION

1. Field of the Invention

The present invention relates to a method and system of verifying a protein-protein interaction.

2. Description of the Related Art

Protein is a material which is generated by the expression of a gene, which performs inherent functions in a living body and plays a leading role for various living organisms while organically interacting with other proteins. For example, a signal transmission for transmitting a bio-signal to a nucleus, thus causing a biological phenomenon to occur, the life period and development of a cell, metabolism, etc. are performed through complicated interactions among a plurality of proteins. Accordingly, contemporary biological science has focused on complicated interactions between genes or proteins, rather than on only individual genes or proteins, in order to investigate life phenomena from a more general view.

A protein-protein interaction may be defined as an interaction involving several proteins for a specific biological process in a living organism. That is, a protein-protein interaction may be understood as an interaction in which a protein reacts with another specific protein. In general, a protein-protein interaction is analyzed through high-throughput screening such as yeast two hybrids. However, the analysis result (data) contains a lot of false positives that are not substantial protein-protein interaction results. A biological test, such as co-immunoprecipitation, may be performed to detect the false positives but is expensive since the scale of protein-protein interactions is very large.

At the present time, a large amount of researches has been conducted into estimation of protein-protein interactions, not verification thereof. Estimation methods of protein-protein interactions are largely categorized into a mechanical learning method and a protein homology method. However, these methods also give many false positives. Therefore, a method of verifying protein-protein interactions must be developed to secure data reliability.

Conventionally, in order to verify protein-protein interactions, a lot of time is required to search a database which includes articles or patent documentation disclosing various bio-information, in order to find a document describing protein using a keyword search engine, and reading the searched document.

However, as the amount of documentation disclosing bio-information has increased exponentially in the field of biology, it is virtually impossible to rapidly and precisely verify information regarding a desired protein-protein interaction according to the above method.

### SUMMARY OF THE INVENTION

The present invention provides a method of rapidly and precisely verifying a protein-protein interaction estimated by a user, based on the existing documents.

The present invention also provides a system for rapidly and precisely verifying a protein-protein interaction estimated by a user, based on the existing documents.

According to an aspect of the present invention, there is provided a method of verifying a protein-protein interaction, the method comprising (a) extracting protein-protein interaction information from protein-related documents searched for from a bio-information document database, according to a text mining method; (b) mapping the protein-protein interaction information to corresponding ontology identifications; and (c) filtering the mapped protein-protein interaction information according to a frequency of the information and an impact factor of a corresponding protein-related document in order to obtain highly-weighted information.

The method may further comprise (d) making an index of information regarding the protein-related documents, protein-related sentences in the documents, the ontology identifications, and the protein-protein interaction information and the reliability thereof.

(a) may comprises (a1) tagging the protein-related documents which include protein-related terms; (a2) extracting sentences related to protein-protein interactions from the tagged documents; and (a3) recognizing from the extracted sentences a subject word regarding a protein, an object word regarding another protein, and an event word representing the relationship between the subject word and the object word.

During (b), the protein-protein interaction information may be mapped to the corresponding ontology identifications according to species of organism, based on an ontology database.

(c) may comprises (c1) when several pieces of protein-protein interaction information conflict each other, computing weights to be given to each of the several pieces of the protein-protein interaction information; and (c2) when the difference between the computed weights is greater than a specific threshold, selecting information having the highest weight from the several pieces of the protein-protein interaction information.

According to another aspect of the present invention, there is provided a system for verifying a protein-protein interaction, the system comprising an ontology database storing information regarding interactions of proteins and a hierarchical structure of the proteins; a text mining unit extracting protein-protein interactions from protein-related documents according to a text mining method; an ontology mapping unit mapping the protein-protein interactions to ontology identifications based on the ontology database; and a filtering unit filtering the mapped protein-protein interaction information according to a frequency of the information and an impact factor of a corresponding protein-related document in order to obtain highly-weighted information.

The system may further comprise an information index unit making an index of information regarding the protein-related documents, protein-related sentences in the documents, the ontology identifications, and the protein-protein interaction information and the precision thereof, and storing the index in an interaction information database.

The text mining unit may performs (a1) tagging the protein-related documents which include protein-related terms; (a2) extracting sentences related to protein-protein interactions from the tagged documents; and (a3) recognizing from the extracted sentences a subject word regarding a protein, an object word regarding another protein, and an event word representing the relationship between the subject word and the object word.

The information filtering unit may performs (c1) computing weights to be given to each of several pieces of the protein-protein interaction information when the several pieces of the protein-protein interaction information conflict with each other; and (c2) selecting information having the highest weight from the protein-protein interaction information when the difference between the weights is greater than a specific threshold.

### BRIEF DESCRIPTION OF THE DRAWINGS

The above and other aspects and advantages of the present invention will become more apparent by describing in detail exemplary embodiments thereof with reference to the attached drawings in which:

FIG. 1 is a flowchart illustrating a method of verifying a protein-protein interaction according to an embodiment of the present invention;

FIG. 2 is a flowchart of operation S200 of FIG. 1 in more detail according to an embodiment of the present invention;

FIG. 3 is a diagram illustrating a hierarchical structure of an ontology database according to an embodiment of the present invention;

FIG. 4 is a flowchart of operation S400 of FIG. 1 in more detail according to an embodiment of the present invention; and

FIG. 5 is a block diagram of a system for verifying a protein-protein interaction according to an embodiment of the present invention.

### DETAILED DESCRIPTION OF THE INVENTION

Hereinafter, exemplary embodiments of the present invention will be described in detail with reference to the accompanying drawings.

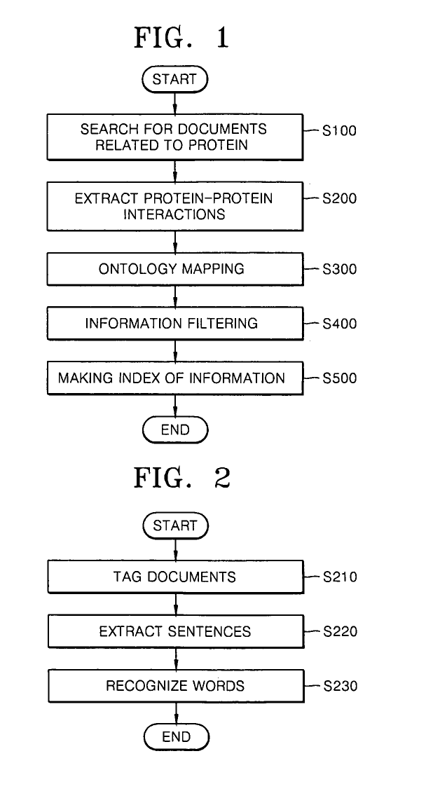

FIG. 1 is a flowchart illustrating a method of verifying a protein-protein interaction according to an embodiment of the present invention. Referring to FIG. 1, the method includes searching a bio-information document database for documents related to protein (S100), extracting protein-protein interactions from the searched documents according to a text mining method (S200), mapping the extracted protein-protein interactions to ontology identifications (ID) (S300), and filtering the protein-protein interaction information to obtain highly-weighted information (S400). Alternatively, the method may further include making an index of information regarding the documents related to protein, protein-related sentences in the documents, the ontology ID, and the protein-protein interactions and the reliability thereof (S500).

The method illustrated in FIG. 1 will now be descried in greater detail.

Searching for Documents Relating to Protein

Protein-related documents are searched for in a bio-information document database in order to verify an estimated protein-protein interaction (S100).

Here, the bio-information document may be a document, such as an article or a patent document, which discloses various bio-information. Operation S100 may be performed by using the conventional keyword engine. The protein-related documents preferably include information regarding protein-protein interactions.

For example, in operation S100, when biologically meaningful names (protein, organisms, a gene, a disease, etc.) are included in documents, an individual name recognition process may be performed to recognize the boundaries of the included terms and determine a category for the meaning of the terms, and documents disclosing protein related to protein-protein interactions may be detected by using the recognized names.

Extraction of Protein-Protein Reaction Information

Next, protein-protein interactions are extracted from the detected documents according to the text mining method (S200).

FIG. 2 is a flowchart illustrating operation S200 of FIG. 1 in more detail according to an embodiment of the present invention. Referring to FIG. 2, operation S200 may include tagging documents (S210), extracting sentences (S220), and recognizing words (S230).

Specifically, in operation S210, tagging the protein-related documents which include protein-related terms is performed. It would be apparent to those of ordinary skilled in the art that various methods can be used to perform tagging on the terms. For example, the terms may be categorized into a noun, a verb, and an adjective, and different tags may be assigned to the categorized terms. For example, terms related to protein may be selected beforehand and when the selected terms are included in a document, a specific tag may be assigned to them. Also, verbs related to chemical interactions, e.g., “bind”, “react”, “activate”, or “inhibit”, may be selected beforehand, and when the selected verbs are included in a document, a predetermined tag may be assigned to them.

In operation S220, the tagged documents are analyzed according to a predetermined logic, and sentences related to protein-protein interactions are extracted from the analyzed result.

In operation S230, a subject word regarding a protein, an object word regarding another protein, and an event word representing the relationship between the subject word and the object word are recognized from the extracted sentences. Through the recognition, protein-protein interactions having a significant biological meaning can be extracted.

Ontology Mapping

A string of words included in a text may have the same meaning even if their formats are slightly different from each other. Also, the string of the words may be differently understood according to the species of organism. To solve this problem, a string of words describing protein and protein-protein interactions must have a controlled vocabulary and meaning system. Accordingly, in the method of verifying a protein-protein interaction according to the present invention, the extracted protein-protein interactions are mapped to ontology ID (S300).

In operation S300, the protein-protein interactions may be mapped to ontology ID according to the species of organism, based on an ontology database. The ontology database may be a well-known gene ontology database, such as “SwissProt” or “GO”.

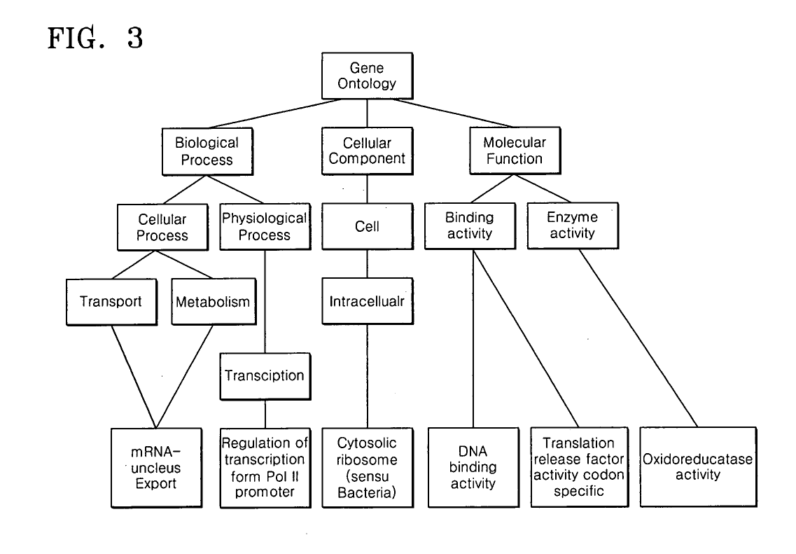

FIG. 3 is a diagram of a hierarchical structure of a gene ontology database according to an embodiment of the present invention. Referring to FIG. 3, the gene ontology database consists of three parts: a cellular component part, a biological process part, and a molecular function part. The gene ontology database may store gene ontology information that is hierarchical information representing the relationship between proteins.

The cellular component part may specify the structure and location of each cell, and a set of giant molecules. The biological process part may consist of combinations of arranged molecular functions, and specify chemical interactions thereof. The molecular function part may specify the functions of individual genes or proteins.

Information Filtering

When processing a large amount of documents, a conflict of information may be caused due to a mechanical processing error or contrary opinions in different documents. To solve this problem, in the method illustrated n FIG. 1, highly-weighted information is obtained by filtered the mapped protein-protein interactions according to the frequency of appearance of a piece of conflicting information and the impact factor of the corresponding protein-related document (S400).

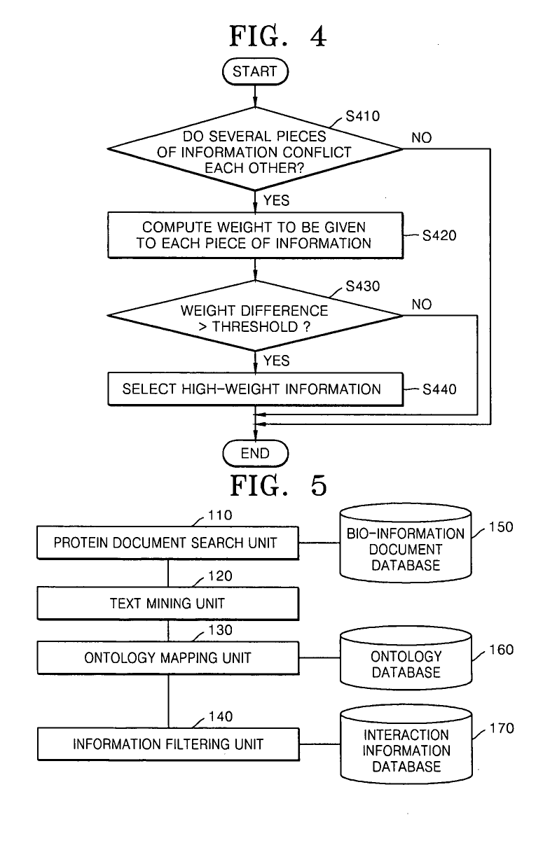

FIG. 4 is a flowchart of operation S400 illustrated in FIG. 1 in more detail according to an embodiment of the present invention. Referring to FIG. 4, when it is determined that several pieces of conflicting information regarding the same protein-protein interaction are found in several documents (S410), a weight to be given to the several pieces of the information is computed (S420). A criterion or a method of computing the weights is not limited. For example, the weights may be computed based on the frequency of appearance of a piece of the conflicting information and the impact factors of documents disclosing a piece of the conflicting information.

Next, if it is determined that the difference between the weights is greater than a specific threshold (S430), the information given the highest weight is selected from the several pieces of the information (S440). That is, the most reliable information is selected from the conflicting protein-protein interaction information. If the difference between the weights is not greater than the specific threshold, that is, when any one piece of the conflicting protein-protein interaction information is not significantly more reliable than the other pieces of information, no information is selected from the conflicting protein-protein interaction information.

Making Index of Information

Alternatively, the method of FIG. 1 may further include making an index of information regarding the documents related to protein, protein-related sentences in the documents, the ontology ID, and the protein-protein interactions and the reliability thereof (S500). The index of the information may be stored in an interaction information database.

FIG. 5 is a block diagram of a system for verifying a protein-protein interaction according to an embodiment of the present invention. Referring to FIG. 5, the system includes an ontology database 160 storing information regarding the relationship among proteins and a hierarchical structure thereof, a text mining unit 120 extracting protein-protein interactions from protein-related documents according to the text mining method, an ontology mapping unit 130 mapping the protein-protein interactions to ontology ID based on the ontology database 160, and an information filtering unit 140 filtering the mapped protein-protein interactions according to the frequency of appearance of the information and an impact factor of the corresponding protein-related document in order to obtain highly-weighted information.

The system may further include an information index unit (not shown) that makes an index of information regarding the protein-related documents, protein-related sentences in the documents, ontology IDs, and protein-protein interactions and the reliability thereof, and stores the index of the information in a interaction information database 170.

The system may further include a bio-information document database 150 that stores bio-documents disclosing various bio-information, and a protein document search unit 110 that searches the bio-information document database 150 for protein-related documents.

The text mining unit 120 may (a1) perform tagging on terms in the protein-related documents, (a2) extract sentences related to protein-protein interactions from the tagged documents, and (a3) perceive from the extracted sentences a subject word regarding a protein, an object word regarding another protein, and an event word representing the relationship between the subject word and the object word.

The information filtering unit 140 may (c1) compute weights to be given to several pieces of conflicting protein-protein interaction information, and (c2) select information having the highest weight from the conflicting information when the difference between the weights is greater than a specific threshold.

The present invention can be embodied as computer readable code in a computer readable medium. Here, the computer readable medium may be any recording apparatus capable of storing data that is read by a computer system, e.g., a read-only memory (ROM), a random access memory (RAM), a compact disc (CD)-ROM, a magnetic tape, a floppy disk, an optical data storage device, and so on. Also, the computer readable medium may be a carrier wave that transmits data via the Internet, for example. The computer readable medium can be distributed among computer systems that are interconnected through a network, and the present invention may be stored and implemented as a computer readable code in the distributed system.

As described above, according to the present invention, it is possible to prevent redundant experiments by utilizing the knowledge supported by existing documents, and check the validity of the experiments, prior to experimental verification of an estimated protein-protein interaction. Also, the result of executing a system that estimates a protein-protein interaction can be verified by using the related documents, thereby evaluating the performance of the system based on the result.

While this invention has been particularly shown and described with reference to preferred embodiments thereof, it will be understood by those skilled in the art that various changes in form and details may be made therein without departing from the spirit and scope of the invention as defined by the appended claims.

What is claimed is:

1. A method of verifying a protein-protein interaction, comprising:
(a) extracting protein-protein interaction information from protein-related documents searched for from a bio-information document database, according to a text mining method;
(b) mapping the protein-protein interaction information to corresponding ontology identifications; and
(c) filtering the mapped protein-protein interaction information according to a frequency of the information and an impact factor of a corresponding protein-related document in order to obtain highly-weighted information.

2. The method of claim 1, further comprising (d) making an index of information regarding the protein-related documents, protein-related sentences in the documents, the ontology identifications, and the protein-protein interaction information and the reliability thereof.

3. The method of claim 1, wherein (a) comprises:
(a1) tagging the protein-related documents which include protein-related terms;
(a2) extracting sentences related to protein-protein interactions from the tagged documents; and
(a3) recognizing from the extracted sentences a subject word regarding a protein, an object word regarding another protein, and an event word representing the relationship between the subject word and the object word.

4. The method of claim 1, wherein during (b), the protein-protein interaction information is mapped to the corresponding ontology identifications according to species of organism, based on an ontology database.

5. The method of claim 1, wherein (c) comprises:
(c1) when several pieces of protein-protein interaction information conflict each other, computing weights to be given to each of the several pieces of the protein-protein interaction information; and
(c2) when the difference between the computed weights is greater than a specific threshold, selecting information having the highest weight from the several pieces of the protein-protein interaction information.

6. A system for verifying a protein-protein interaction, comprising:
an ontology database storing information regarding interactions of proteins and a hierarchical structure of the proteins;
a text mining unit extracting protein-protein interactions from protein-related documents according to a text mining method;
an ontology mapping unit mapping the protein-protein interactions to ontology identifications based on the ontology database; and
a filtering unit filtering the mapped protein-protein interaction information according to a frequency of the information and an impact factor of a corresponding protein-related document in order to obtain highly-weighted information.

7. The system of claim 6, further comprising an information index unit making an index of information regarding the protein-related documents, protein-related sentences in the documents, the ontology identifications, and the protein-protein interaction information and the precision thereof, and storing the index in an interaction information database.

8. The system of claim 6, wherein the text mining unit performs:
(a1) tagging the protein-related documents which include protein-related terms;
(a2) extracting sentences related to protein-protein interactions from the tagged documents; and
(a3) recognizing from the extracted sentences a subject word regarding a protein, an object word regarding another protein, and an event word representing the relationship between the subject word and the object word.

9. The system of claim 6, wherein the information filtering unit performs:
(c1) computing weights to be, given to each of several pieces of the protein-protein interaction information when the several pieces of the protein-protein interaction information conflict with each other; and
(c2) selecting information having the highest weight from the protein-protein interaction information when the difference between the weights is greater than a specific threshold.
